(19) United States
(12) Patent Application Publication
(10) Pub. No.: US 2025/0345549 A1
(43) Pub. Date: Nov. 13, 2025

(54) NO SUPPLY APPARATUS SUITABLE FOR LAND OR AIR TRANSPORT

(71) Applicant: INOSYSTEMS, Antony (FR)

(72) Inventors: Frederic MARCHAL, Antony (FR); Thierry BOULANGER, Antony (FR); Bogdan A ARAMA, Antony (FR); Mary SCHMITT, Antony (FR); Yann BLANDIN, Antony (FR)

(21) Appl. No.: 19/190,376

(22) Filed: Apr. 25, 2025

(30) Foreign Application Priority Data
May 13, 2024 (FR) 2404880

(51) Int. Cl. A61M 16/12; A61M 16/00; A61M 16/20

(52) U.S. Cl. CPC A61M 16/12; A61M 16/0051; A61M 16/203; A61M 2016/0033; A61M 2202/0275; A61M 2205/505

(57) ABSTRACT

The invention relates to an apparatus (1) for delivering NO-containing gas, comprising an internal gas circuit (200) with flowrate control means (220, 230, 240) for controlling the flow of gas, in particular a proportional solenoid valve (220) and a flowrate measurement device (230). Operating means (210) cooperate with the flowrate control means (220, 230, 240) to authorize, prohibit or adjust the flowrate of gas within the gas circuit (200). The flowrate measurement device (230) comprises a bidirectional flowrate sensor configured to perform positive and negative flowrate measurements of the NO-containing gas flow circulating in the gas circuit (200), and to supply said flowrate measurements to the operating means (210). Installation (100) for supplying NO-containing gas, comprising such an NO delivery apparatus (1).

### CROSS-REFERENCE TO RELATED APPLICATIONS

This patent application claims priority to French Application No. 2404880, filed May 13, 2024 and the entire contents of which are incorporated herein by reference.

### TECHNICAL FIELD

The invention relates to an NO delivery apparatus well suited for land or air transport, and to an installation for supplying an NO-based gas mixture to a patient, typically an NO/nitrogen (N₂) mixture, comprising such an NO delivery apparatus and a medical ventilator delivering an oxygen-based respiratory gas (i.e. ≥ approximately 20%).

### BACKGROUND

Inhaled nitric oxide (i.e. NO or iNO) is a gaseous medicament commonly used to treat patients suffering from acute pulmonary arterial hypertension, in particular pulmonary vasoconstrictions in adults or children, including the newborn (PPHN), as described for example in EP-A-560928 or EP-A-1516639.To implement therapy by inhaled NO, use is made of a gas supply installation, also called an NO administration installation, comprising an NO delivery apparatus and a medical ventilator, that is to say a respiratory assistance apparatus, supplying a patient circuit that generally comprises one or more flexible ducts which are fluidically connected to a respiratory interface, such as a tracheal intubation tube or the like, serving to deliver to the patient to be treated a final gas mixture containing NO.

A gas supply installation of this kind is described, for example, in EP 3821929. This type of installation is used in a hospital environment to administer the treatment by NO and thereby to care for patients who need to inhale NO in order to treat their pulmonary arterial hypertension. Installations of this type are also described in EP 3233171, EP 3410927, EP 4209243, EP 4241817, EP 4241812 and EP 4295882.

In such an installation, the NO delivery apparatus makes it possible to inject a gas flow based on NO, typically an NO/nitrogen mixture, into the patient circuit, which is also supplied with a respiratory gas flow containing oxygen (at least approximately 20 vol %), such as air or an oxygen/nitrogen mixture (O₂/N₂), supplied by the medical ventilator, so as to obtain a combined flow, also called the “final gas mixture”, comprising NO at the desired dosage, typically less than 80 ppmv of NO, at least approximately 20 vol % of oxygen and nitrogen (N₂), and even some unavoidable impurities.

Means for controlling the flowrate in the apparatus make it possible to control or adjust the flowrate of gas containing NO, e.g. NO/N₂ mixture, in order to obtain the desired combined gas mixture, i.e. the final mixture containing NO at the desired dosage.

Such control means generally comprise one or more actuators such as a proportional solenoid valve and a flowrate sensor, which are operated by an electronic controller or the like, that is to say operating means, so as to make it possible to generate different values of NO flowrates as a function of the concentration set by the physician, i.e. the desired dosage, and of the NO concentration of the gas supplied by the one or more cylinders of NO/N₂ mixture.

A patient who requires NO must be able to be supplied with the latter in all circumstances, i.e. whether the patient is static, for example bedridden within a hospital, or in motion, i.e. during possible transport of the patient, regardless of whether this transport takes place within the hospital itself, for example on a stretcher, a mobile bed or the like, or in a land or air transport vehicle, such as an ambulance, an emergency services vehicle or the like, or in an aircraft, such as an aeroplane or a helicopter.

In all cases, it is necessary to be able to regulate and therefore deliver flowrates over a wide range of flowrates, typically between 0 and 10 l/min, in order to cover dosages ranging from 1 to 80 ppm of NO, in particular for an NO concentration of the gas supplied by the cylinder or cylinders which is less than 1500 ppmv and usually between 200 and 1000 ppmv.

Consequently, in order to perform precise regulation of the NO flowrate between a few ml/min and approximately 10 l/min, it is necessary to use one or more very precise NO flowrate sensors, that is to say typically with a precision or resolution of below 1 ml/min.

However, the more sensitive the flowrate sensor is, the more it will also sense environmental disturbances, in particular shocks and/or vibrations, that may occur during transport of the patient within the hospital, and even more notably when the patient is in a land or air transport vehicle.

However, these environmental disturbances, i.e. shocks and/or vibrations, can lead to saturation of the signal by “falsifying” the mean value of the signal, which has a negative impact on the regulation of the NO flowrate, with the risk of untimely closing of the proportional solenoid valve providing the NO-based gas flowrate, hence prematurely stopping the delivery of NO to the patient, causing a negative rebound effect in some patients. However, for obvious health safety reasons, such a rebound effect is obviously undesirable for patients.

WO2015168517 discloses an installation for supplying an NO/air mixture, including an NO/N₂ supply apparatus comprising a duct for conveying the NO/N₂ mixture, on which duct a valve and a flowrate sensor are arranged. The valve can be of various types, namely a proportional valve, an all-or-nothing valve or the like. The flowrate sensor is unidirectional. It serves to measure the flowrate of NO/N₂ mixture only in the positive direction, i.e. only in the direction of the injector arranged on the patient circuit fed by the medical ventilator. A second sensor, which is of the bidirectional type, is arranged in the injector itself, hence outside the apparatus, and serves to monitor the positive and negative flows coming from the ventilator, which delivers an oscillating air flow.

In addition, US2023270960 discloses an installation similar to that of WO2015168517, including a bidirectional sensor arranged on the air/O₂ line in order to detect a possible negative flow of the air/O₂ flow and, if appropriate, to trigger an alarm.

None of these documents addresses the measurement problems caused by environmental disturbances, such as vibrations, occurring during transport of the NO delivery apparatus, and the resulting problem of poor regulation of the supply of NO, i.e. typically an NO/N₂ gas mixture.

A problem is therefore to improve patient safety by proposing an improved NO delivery apparatus that is less sensitive to the environmental disturbances, i.e. shocks and/or vibrations, that may cause signal saturation during transport of the patient within a hospital or while in a land or air transport vehicle.

### SUMMARY

One solution concerns an apparatus for delivering, i.e. supplying, a gas containing NO, such as an NO/N₂ gas mixture, comprising: an internal gas circuit for conveying a flow of NO-containing gas, comprising flowrate control means configured to control the flowrate of NO-containing gas within the gas circuit, said flowrate control means comprising a proportional solenoid valve and a flowrate measurement device, and operating means cooperating with the flowrate control means to authorize, prohibit or adjust the gas flowrate within said gas circuit.

Moreover, according to the invention, the flowrate measurement device comprises a bidirectional flowrate sensor configured to perform positive and negative flowrate measurements of the NO-containing gas flow circulating in the gas circuit, and to supply said flowrate measurements to the operating means, said operating means processing the positive and negative flowrate measurements coming from the flowrate measurement device in order to operate the proportional solenoid valve.

Depending on the embodiment considered, the apparatus of the invention can comprise one or more of the following features:
the operating means are configured to operate the proportional solenoid valve on the basis of an average flowrate value taking into account the negative flowrates supplied by the bidirectional flowrate sensor.
the flowrate measurement device is arranged in the gas circuit upstream of the proportional solenoid valve, considering the direction of flow of the gas.
the operating means comprise at least one microprocessor.
the proportional solenoid valve and the flowrate measurement device form all or part of a mass flow controller (MFC).

Moreover, depending on the embodiment considered, the apparatus of the invention can comprise one or more of the following additional features:
it comprises NO dose-setting means configured to allow a user to set a desired dose of NO, i.e. a dosage.
the NO dose-setting means are configured to allow a user to set an NO dose of between 1 and 80 ppmv, generally an NO content setpoint of between 1 and 50 ppmv, typically between 5 and 40 ppmv, or another dose.
the operating means cooperate with the NO dose-setting means.
the NO dose that is set is supplied to the operating means.
it comprises a graphical display or any other display device, such as a touch screen.
the operating means are configured to order the display, on the graphical display, of the NO dose that is set.
the operating means are configured to order the display, on the graphical display, of high and low NO alarm thresholds determined by the operating means as a function of the NO dose that is set.
the internal gas circuit of the apparatus conveying the NO-containing gas, such as an NO/N₂ mixture, is fluidically connected to a respiratory circuit conveying a respiratory gas containing oxygen, such as air or an O₂/N₂ gas mixture (>20% O₂), in order to inject the NO-containing gas into it and obtain a combined gas mixture containing NO and oxygen, that is to say the final mixture to be administered to the patient.
it comprises NO concentration measurement means for determining the NO content in the combined gas mixture containing NO and oxygen, such as an NO sensor, for example with an electrochemical cell.
it comprises alarm means, typically an acoustic and/or visual alarm.
the operating means are configured to act on alarm means in order to trigger an alarm, in particular an acoustic and/or visual alarm.
the alarm means can comprise a buzzer-type device, and/or a loudspeaker, or any other device for emitting an acoustic signal that can be heard by the human ear, i.e. by the medical personnel.
the alarm means can comprise a luminous device, for example comprising one or more diodes (LEDs) or the like.
the alarm means can comprise the display of an alert message or similar on the graphical display.
the operating means are configured to order a display, on the graphical display, of the NO content in the combined gas mixture that has been determined by the NO concentration measurement means.
the dose-setting means are configured to allow the user to modify the NO dose (D_{NO}) and/or the high and low NO alarm thresholds.
it is fed with a gas containing NO in a given initial proportion (e.g. an NO/N₂ mixture), in general an initial proportion of NO of between 100 and 1500 ppmv, typically between 200 and 1000 ppmv.
it is supplied with a gaseous mixture formed of nitrogen and NO.
the NO dose-setting means are configured to allow the user to set an NO dose, i.e. NO content setpoint, corresponding to the desired proportion of NO in the combined gas mixture, i.e. after mixing of the NO/N₂ mixture with the oxygen-based respiratory gas flow, i.e. an NO dosage.
the NO dose-setting means are part of an HMI (human-machine interface) or GUI (graphical user interface).
the HMI comprises the graphical display, preferably with colour display.
the NO dose-setting means comprise one or more virtual touch keys or the like, which can be actuated by the user, displayed on the graphical display of the digital touch screen type with touch panel.
it comprises storage means.
the storage means comprise a computer memory, for example a flash memory, RAM or the like.
the flowrate control means are arranged on the internal gas circuit.
the mass flow controller or MFC comprises at least the proportional solenoid valve operated by the operating means and the flowrate sensor.
the operating means comprise one or more (micro) processors arranged on one or more electronic boards.
the operating means form, are or comprise a (micro) controller or the like.
the operating means comprise one or more (micro) processors implementing one or more algorithms, in particular one or more algorithms for operating the flowrate control means, one or more algorithms for processing the flowrate measurements and/or one or more algorithms for processing the NO, NO₂ and/or O₂ concentration measurements, etc.
the storage means are arranged on the electronic board.

The invention also relates to an installation for supplying NO-containing gas, comprising the NO delivery apparatus according to the invention fed with an NO/N₂ gas mixture by one or more NO/N₂ mixture sources, and a medical ventilator configured to supply a flow of respiratory gas containing O₂.

Depending on the embodiment considered, the installation of the invention for supplying NO-containing gas can comprise one or more of the following features:
the medical ventilator is configured to supply a flow of respiratory gas containing at least 20 vol % approximately of O₂, typically an NO/N₂ mixture or air.
the NO delivery apparatus and the medical ventilator are fluidically connected to a respiratory circuit in order to supply it with the flow of NO-containing gas (e.g. NO/N₂) and the flow of oxygen-containing gas (e.g. air or O₂/N₂) and thereby obtain a combined gas mixture to be supplied to the patient, for example via a tracheal intubation tube or the like.
a flowrate sensor is arranged in the respiratory circuit between the medical ventilator and the injection device.
an injection device is arranged in the respiratory circuit, downstream of the flowrate sensor.
the injection device is configured to inject the NO-containing gas from the NO delivery apparatus into the flow of O₂-containing respiratory gas from the medical ventilator in order to obtain the combined gas mixture to be supplied to the patient needing it.
the injection device is configured to mix the NO-containing gas from the NO delivery apparatus with the flow of O₂-containing respiratory gas conveyed through the respiratory circuit, and to obtain a combined gas mixture containing NO and oxygen, or even other compounds such as nitrogen or unavoidable impurities.
the injection device comprises a first gas inlet supplied with a flow of O₂-containing respiratory gas, i.e. from the medical ventilator.
the injection device also comprises a second gas inlet supplied with NO-containing gas from the NO delivery apparatus.
the injection device also comprises a gas outlet supplying the combined gas mixture containing NO and oxygen, obtained by mixing, within the injection device, the gas containing NO (e.g. NO/N₂ mixture) with the flow of respiratory gas containing O₂ (e.g. air or O₂/N₂ mixture).
the medical ventilator delivers air or an oxygen/nitrogen mixture, i.e. as a respiratory gas containing at least 20 vol % approximately of oxygen, preferably at least 21 vol % approximately of oxygen.
the medical ventilator comprises a motorized blower (i.e. turbine, compressor or similar) delivering the respiratory gas, typically air or an oxygen/nitrogen mixture or, according to another embodiment, an internal gas circuit comprising one or more proportional valves for conveying the gas and controlling its supply, in particular its flowrate. Such a ventilator is generally supplied with respiratory gas via one or more wall outlets supplied with gas from a network of channels in a hospital establishment or building, typically with air or an oxygen/nitrogen mixture.
the medical ventilator comprises control means or a control device, such as one or more electronic control boards. Preferably, the control means of the medical ventilator operate or control the motorized blower or, depending on the circumstances, the proportional valves of the medical ventilator.
the medical ventilator is of the HFO type or comprises an HFO function, that is to say it is able to produce high-frequency oscillations.
the NO source(s) supplying the NO delivery apparatus contains a gaseous mixture NO/N₂ containing between 100 and 2000 ppmv of NO, the remainder being nitrogen (N₂), preferably between 100 and 1000 ppmv of NO, preferably at a pressure of between 10 and 250 bar abs, typically at over 100 bar abs (before start of withdrawal).
the NO source(s) is or comprises one or more gas cylinders, preferably having a capacity of between 0.5 and 50 | (water equivalent).
the gas cylinder(s) comprises a cylindrical body made of steel or aluminium alloy and is equipped with a simple valve (without regulator) or a valve with an integrated regulator or IRV, preferably an IRV protected by a protective cap, for example made of metal or polymer.
the respiratory circuit (i.e. patient circuit) comprises an inspiratory branch and an expiratory branch, typically flexible ducts forming the inspiratory branch and the expiratory branch, for example polymer hoses.
the inspiratory branch and the expiratory branch, e.g. flexible ducts, are connected to a joining piece, such as a Y-piece.
the inspiratory branch and/or the expiratory branch are fluidically connected to a patient respiratory interface, preferably via the joining piece.
the patient respiratory interface comprises a tracheal intubation tube or a breathing mask or the like.
the respiratory circuit, in particular the inspiratory branch, comprises a gas humidifier.
the gas humidifier is arranged downstream of the injection device, for example an NO injection module, so as to be able to humidify the gas before the latter is administered to the patient by inhalation.
the ventilator and the NO delivery apparatus are supplied electrically by one or more electric current sources, typically the mains supply (110/220 V) and/or one or more rechargeable batteries.

According to another aspect, the invention also relates to a method for therapeutic treatment of a person, i.e. a human patient (i.e. adult, child, adolescent or neonate), suffering from pulmonary hypertension and/or hypoxia, which cause pulmonary vasoconstriction or similar, said method comprising administration by inhalation, to the person requiring it, of a gaseous mixture comprising from 1 to 80 ppmv of NO, typically less than 40 ppmv, and approximately at least 20 vol % of oxygen, preferably approximately at least 21 vol % of oxygen, by means of a gas supply installation, as described above according to the invention, comprising an NO delivery apparatus according to the invention for delivering NO, so as to treat (at least partially) said pulmonary hypertension and/or said hypoxia, which can be caused by one or more pulmonary diseases or disorders such as PPHN (persistent pulmonary hypertension of the newborn) or ARDS (acute respiratory distress syndrome) or can be caused by heart surgery with placement of the patient on extracorporeal blood circulation.

### Definitions

In general, within the context of the invention:
“ppmv” means parts per million by volume.
“vol %” means percentage by volume.
“NO” denotes nitric oxide.
“NO₂” denotes nitrogen dioxide.
“N₂” denotes nitrogen.
“O₂” denotes oxygen.
the terms “concentration”, “quantity”, “proportion”, “dose” and “content” are considered as equivalents.
the terms “means of/to/for” are considered to be wholly equivalent to and capable of being substituted by the terms “device of/to/for”, for example the term “operating means” may be replaced by “operating device”, the term “valve means” may be replaced by “valve device”, the term “storage means” may be replaced by “storage device”, etc.
“flowrate measurement” is understood as a flowrate value (e.g. a numerical value) or a signal representative of such a flowrate value, which reflects or corresponds to a gas flowrate measured by a flowrate sensor, such as a mass flow sensor.
“pressure measurement” is understood as a pressure value (e.g. a numerical value) or a signal representative of such a pressure value, which reflects or corresponds to the gaseous pressure measured by a pressure sensor.
“concentration measurement” is understood as a value for the content of a given gaseous compound, such as NO, NO₂ or O₂, that is to say a numerical value or a signal representative of such a content value, which reflects or corresponds to the proportion (i.e. quantity) of the gaseous compound considered in a given gas mixture, measured by concentration measurement means, such as electrochemical cell sensors.

### BRIEF DESCRIPTION OF VIEWS OF DRAWINGS

The invention will now be better understood from the following detailed description, which is given by way of non-limiting illustration and with reference to the appended figures, in which:

FIG. 1 shows schematically an embodiment of a gas administration installation comprising an NO delivery apparatus according to the invention.

FIG. 2 shows schematically an embodiment of the internal architecture of the NO delivery apparatus according to the invention, such as that of the installation of FIG. 1.

FIG. 3 shows a flowrate curve obtained with a conventional unidirectional sensor, in the absence of any vibration.

FIG. 4 shows a flowrate curve obtained with a conventional unidirectional sensor, in the presence of vibrations, such as those that occur during transport of the patient.

FIG. 5 shows a flowrate curve obtained in the presence of vibrations, but with a special sensor according to the present invention.

### DETAILED DESCRIPTION

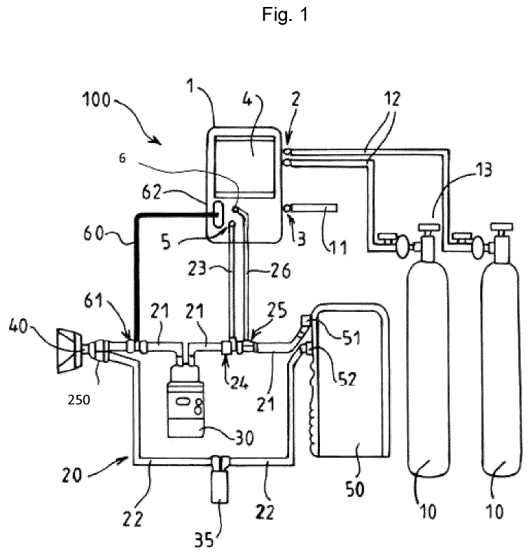

FIG. 1 shows schematically an embodiment of a gas administration installation 100 according to the invention, comprising an NO delivery apparatus 1 according to the invention for supplying a gaseous mixture based on nitric oxide (NO), typically an NO/N₂ gas mixture, and a medical ventilator which supplies a gas containing at least 20 vol % of oxygen, typically air, an O₂/N₂ mixture or the like.

In this case, the installation 100 comprises two pressurized gas cylinders 10, each containing an NO-based gas mixture, namely an NO/N₂ gas mixture containing in this case between 100 and 1500 ppmv of NO (remainder N₂), for example 450 or 800 ppmv of NO (remainder N₂), or any other suitable concentration, which feed an NO/N₂ mixture to the device or apparatus 1 for delivering or supplying NO, making it possible to monitor and control the supply of the NO/N₂ gas mixture.

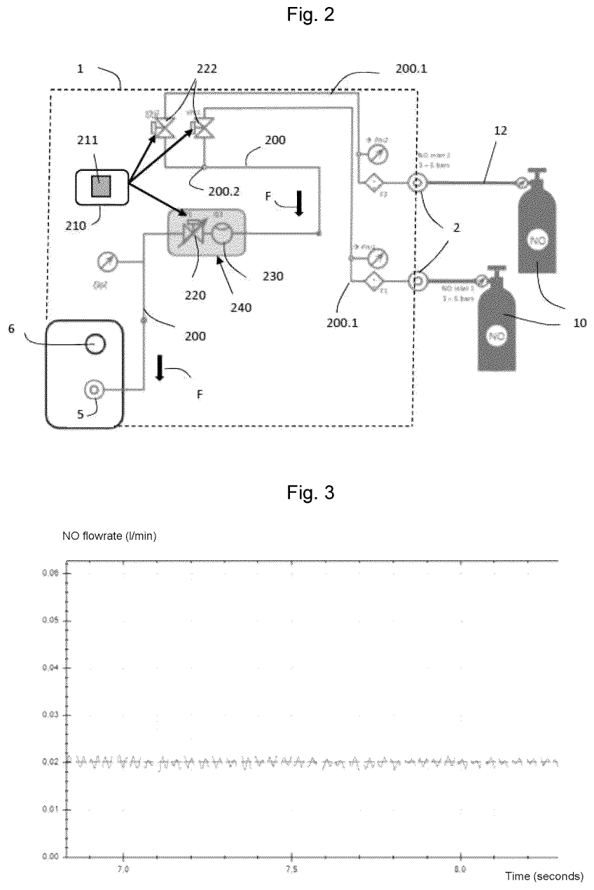

The gas cylinders 10 are fluidically connected to the NO supply apparatus 1 via gas feed lines 12, such as flexible ducts or hoses or the like, which are equipped with devices for setting and/or monitoring the gas pressure, such as gas pressure reducer 13, pressure gauges, etc. The gas feed lines 12 are connected to one or more gas inlets 2 of the NO delivery apparatus 1, which supply an internal gas circuit 200, as shown schematically in FIG. 2, used to convey the gas within the NO supply apparatus 1, i.e. in the casing or shell of the NO delivery apparatus according to the invention.

In the embodiment of FIG. 2, the internal gas circuit 200 is connected to two gas inlets 2 arranged in parallel and each feeding a dedicated inlet section 200.1 of the internal gas circuit 200. Control valves 222 or the like operated by operating means 210 of the apparatus 1 control the passage of the NO/N₂ flow in these inlet sections 200.1 and in the rest of the circuit 200. These two sections 200.1 meet at a connection site 200.2 of the circuit 200, which is situated downstream of the control valves 222.

The NO delivery apparatus 1 also comprises an oxygen inlet 3, which is fluidically connected, via an oxygen feed line 11 such as a flexible hose or the like, to an oxygen source (not shown), for example a pressurized oxygen cylinder or a hospital network. This makes it possible to feed the internal gas circuit 200 with oxygen when necessary.

The medical ventilator 50, i.e. a respiratory assistance apparatus, supplies a flow of oxygen-based respiratory gas, i.e. containing approximately at least 20 vol % of oxygen, preferably approximately at least 21 vol % of oxygen, such as air or an oxygen/nitrogen mixture (N₂/O₂). The medical ventilator 50 and the NO supply apparatus 1 of the installation 100 are in fluidic communication with a respiratory circuit 20, also called the patient circuit, in particular with a gas feed line or inspiratory branch 21, which serves to convey the gas flow to the respiratory interface 40 supplying the gas flow to the patient, that is to say a combined gas mixture, i.e. final mixture, containing the desired dosage of NO. This combined gas mixture is obtained by mixing the oxygen-based flow (e.g. air or O₂/N₂ mixture) from the medical ventilator 50 and the NO-containing flow, i.e. the NO/N₂ gas mixture, delivered by the NO delivery apparatus 1.

For this purpose, the NO delivery apparatus 1 supplies or injects the NO/N₂ mixture into the respiratory circuit 20, conveying the oxygen-based flow, via an injection channel or line 23, which fluidically connects the outlet port 5 of the internal gas circuit 200 of the NO supply apparatus 1 to an injection device 24 arranged on the gas feed line 21.

The injection device 24 is configured to mix the NO-containing gas from the NO delivery apparatus 1 with the flow of O₂-containing respiratory gas coming from the ventilator 50 and conveyed by the inspiratory branch 21 of the respiratory circuit 20, and to obtain a combined gas mixture containing NO and oxygen, i.e. the final gas mixture to be administered to the patient, containing essentially NO at the desired dosage, nitrogen (N₂) and oxygen (O₂), and possibly inevitable impurities (e.g. argon, CO₂, NO₂, etc.), that is to say a final NO/N₂/O₂ gas mixture.

The inspiratory branch 21 further comprises a gas humidifier 30 arranged downstream from the injection device 24, making it possible to humidify the final gas flow, e.g. the combined NO/N₂/O₂ gas mixture, before it is administered by inhalation to the patient to be treated, by means of a respiratory interface 40, such as a tracheal intubation tube, a breathing mask or the like.

A line for recovering the gases exhaled by the patient forms an expiratory branch 22 of the patient circuit 20. It is fluidically connected to the inspiratory branch 21 via a connection piece 250, such as a Y-piece.

At its upstream end, the inspiratory branch 21 is fluidically connected to an outlet port 51 of the medical ventilator 50, such as a connector, coupling or the like, so as to recover and convey the oxygen-based gas, typically air or N₂/O₂ mixture, supplied by the medical ventilator 50, while the expiratory branch 22 conveying the exhaled gases is fluidically connected to an inlet port 52 of the medical ventilator 50, such as a connector, coupling or the like, so as to return to the medical ventilator 50 all or part of the flow of the gases exhaled by the patient.

Furthermore, a flowrate sensor 25, for example of the hot wire or pressure differential type, is arranged on the respiratory circuit 20, in particular on the inspiratory branch 21, between the ventilator 50 and the injection device 24. The flowrate sensor 25 is connected to a connection port 6 of the NO delivery apparatus 1 via one or more flowrate measurement lines 26, which connect to said port 6. It is used to measure the flowrate of gas delivered by the ventilator 50, such as air or N₂/O₂, circulating in the inspiratory branch 21, upstream from the injection device.

These flowrate measurements performed by the flowrate sensor 25 make it possible to control or regulate more efficiently the delivery of the flow of NO (i.e. N₂/O₂) by the NO delivery apparatus 1, in particular the flowrate of NO, since the flowrate measurements performed by the flowrate sensor 25 are returned, via the flowrate measurement line 26 (i.e. electric cables or the like) and the connection port 6, to operating means 210 based on a (micro) processor 211, typically a controller, which process these flowrate measurements.

During the operation of the apparatus 1, the NO/N₂ flow passes through the internal gas circuit 200 in the direction from the gas inlets 2 to the outlet port 5, that is to say to the patient P (i.e. direction of the arrow F in FIG. 2).

In addition, a proportional solenoid valve 220 operated by the operating means 210 is arranged on the internal gas circuit 200 and serves to control or adjust the gas flow circulating there in the direction of the outlet port 5 and the injection line 23, that is to say towards the injection device 24, during normal operation of the apparatus 1.

More precisely, the proportional solenoid valve 220 is associated with a flowrate sensor 230 so as to form a mass flow controller 240 or MFC.

The proportional solenoid valve 220 and the flowrate sensor 230 cooperate with the operating means 210, that is to say one or more operating devices or (micro) controllers. In particular, the flowrate sensor 230 measures the flowrate of NO-containing gas circulating in the circuit 200 and returns the measurements to the operating means 210 and those, in response to these flowrate measurements, control the opening or closing of the proportional solenoid valve 220 in order to adjust the flowrate of gas passing through said proportional solenoid valve 220 in the direction of the outlet port 5.

As is explained below, the flowrate sensor 230 used in the context of the invention is a special sensor, namely a bidirectional flowrate sensor configured to enable measurement of positive and negative flowrates, that is to say in the direction of and in the direction counter to the normal flow (i.e. flow to the patient).

Furthermore, the operating means 210 comprise one or more electronic boards comprising one or more microprocessors 211 implementing one or more algorithms.

The operating means 210 make it possible in particular to adjust or control the flowrate of NO-based gas within the circuit 200, including within the sections 200.1, by controlling the valves 222 and the proportional solenoid valve 220, typically to open or close one or more (solenoid) valves, so as to obtain a given flowrate of NO-based gas, that is to say to permit or stop all or part of the gas flow.

Of course, the operating means 210 also make it possible to process information, measurements or the like, to carry out calculations and/or to control or command other electromechanical elements of the apparatus 1, such as the displays, etc.

In particular, the operating means 210 can determine the NO flowrate to be supplied in order to obtain the desired NO content in the combined mixture, i.e. the desired dosage of NO, in particular on the basis of an NO content set and/or fixed by the user, that is to say an NO dose set by the user, the composition of the NO/N₂ gas mixture supplied by the NO cylinders 10, in particular the NO content in this NO/N₂ mixture, which content can be stored by the apparatus 1, and flowrate measurements carried out by the flowrate sensor 25 arranged on the inspiratory branch 21 and connected by a flowrate measurement line 26 to the operating means 210 via the port 6 for connection to the flowrate sensor 25.

In addition, the gas circuit 200 of the NO supply apparatus 1 can also comprise other elements or components, in particular one or more additional pressure sensors, one or more additional flowrate sensors or flow meters, and/or calibrated-orifice devices or the like (not shown). It can also comprise an emergency line (not shown) which can be used, for example, in the event of a malfunction of the solenoid valve 220 and which bypasses the solenoid valve 220.

The NO delivery apparatus 1 also comprises a graphical user interface (GUI) comprising a graphic display screen 4, preferably a touch screen, i.e. a touch panel, serving to display various information items or data, icons, curves, alerts, etc., and also selection means such as virtual selection keys and/or panes or windows, in particular for making choices, selections or for entering information, such as desired values (e.g. flowrate, dosage of NO, etc.), or any other information or data useful to the healthcare personnel. The display is preferably in colour.

However, according to another embodiment, the apparatus 1 can also comprise one or more mechanical, i.e. non-virtual, selection elements, such as one or more selection buttons or keys actuatable by digital pressure exerted by the user, one or more rotary selection buttons or the like. The selections or choices made via this or these mechanical selection elements can be displayed on the graphical display 4 of the GUI.

The electrical power for the NO supply apparatus 1, in particular for the components requiring electrical current in order to operate, such as the operating means 210, the graphical display 4, etc., is provided conventionally by an electrical current source and/or electrical supply means (not shown), for example a connection to the mains current (110/220V), such as an electrical cord and connection socket, and/or one or more electric, preferably rechargeable, batteries, and/or a current transformer. The electrical power supply to the medical ventilator 50 is ensured in a similar manner, in particular by a connection to the mains current or by an internal battery.

In addition, the installation 100 also comprises a gas sampling line 60 which fluidically connects the inspiratory branch 21 to the NO supply apparatus 1.

The gas sampling line 60 serves to monitor the composition of the combined gas mixture containing NO, O₂ etc., in order to ensure that the contents of NO, O₂ and also toxic NO₂ species which can form are in conformity, in particular to ensure that the proportion of NO is well within the fixed high and low NO alarm thresholds, i.e. between maximum and minimum acceptable NO contents. If this is not the case, the operating means of the apparatus 1 trigger an acoustic alarm and/or a visual alarm in order to alert the healthcare personnel.

The gas sampling line 60, also called the monitoring line, is fluidically connected, at a connection site 61 in FIG. 1, to the gas feed line 21, between the humidifier 30 and the joining piece 250, i.e. the Y-piece, typically in immediate proximity to the joining piece 25, and also to an inlet port 62 of the NO supply device 1, for example a port 62 carried by a connector, coupling or the like, allowing the connection of the gas sampling line 60, such as a flexible hose or the like. The gas samples taken are analysed within a calibration line comprising NO₂, NO and O₂ sensors, such as electrochemical cells forming an internal gas analyser (not shown) electrically connected to the operating means 210. In fact, for safety reasons, it should be verified that the composition of the final gas, i.e. the combined gas mixture, conforms with that of the desired NO/N₂/O₂ gas mixture to be administered to the patient, in particular in order to ensure that it does not contain excessive amounts of toxic NO₂ species, that its oxygen content is not hypoxic, and that its NO content corresponds to the desired dosage, i.e. the dose of NO to be administered by inhalation, which is usually chosen by the healthcare personnel, i.e. a physician or the like.

The desired amount of NO present in the combined mixture administered to the patient, which is typically between 1 and 80 ppmv, i.e. the NO setpoint or dosage, can be entered and/or adjusted and/or modified by the user, preferably via the HMI, by virtue of dose-setting means or the like, such as one or more keys, rotary knobs, cursors or the like, for example one or more virtual keys displayed on the display screen 4. For example, they comprise a “+” key to increment an NO dose value and a “−” key to decrement an NO dose value. Typically, the dose value is incremented or decremented by +/−1 ppmv. Similarly, the NO content in the NO/N₂ gas mixture coming from the cylinders 10 may or may not be adjustable, that is to say is stored once and for all or is able to be set by the user, within a dedicated menu, for example by once again using the setting means.

As has already been explained, a patient requiring NO must be able to be supplied with the latter in all circumstances, i.e. whether the patient is static, for example bedridden within a hospital, or in motion, that is to say during possible transport of said patient. It is then necessary to be able to regulate and deliver NO flowrates over a wide range of flowrates, typically between 0 and 10 l/min, in order to cover dosages ranging from 1 to 80 ppm of NO.

This requires the use of a very precise NO flowrate sensor 230, typically with a precision or resolution of below 1 ml/min.

For this purpose, use is conventionally made of a “conventional” unidirectional flowrate sensor, which is designed to measure the flowrate only in one direction of flow of the gas, namely in the direction of the patient (cf. the arrow F in FIG. 2). Typically, such a unidirectional flowrate sensor measures the gas flowrate using only the positive part of the flowrate signal.

The flowrate sensor 230 of the MFC 240 can be of the pressure differential type, mass type or another type, and cooperates with the operating means 210 to provide them with flowrate measurements of the NO/N₂ flow.

However, the more sensitive the flowrate sensor 230 is, the more it will also sense environmental disturbances, in particular shocks and/or vibrations, that may occur during transport of the patient within the hospital, and even more notably when the patient is in a land or air transport vehicle, such as an ambulance or helicopter.

The problem is that these environmental disturbances, i.e. shocks and/or vibrations, can lead to saturation of the signal by “falsifying” the mean value of the NO flowrate signal (i.e. flow of NO/N₂ mixture) measured by the flowrate sensor 230, which has a negative impact on the regulation of the NO flowrate by control of the proportional solenoid valve 220 by the operating means 210, with the risk of untimely closing of the proportional solenoid valve 220 of the MFC 240 providing the NO-based gas flowrate, hence prematurely stopping the delivery of NO to the patient, causing a negative rebound effect in the patient.

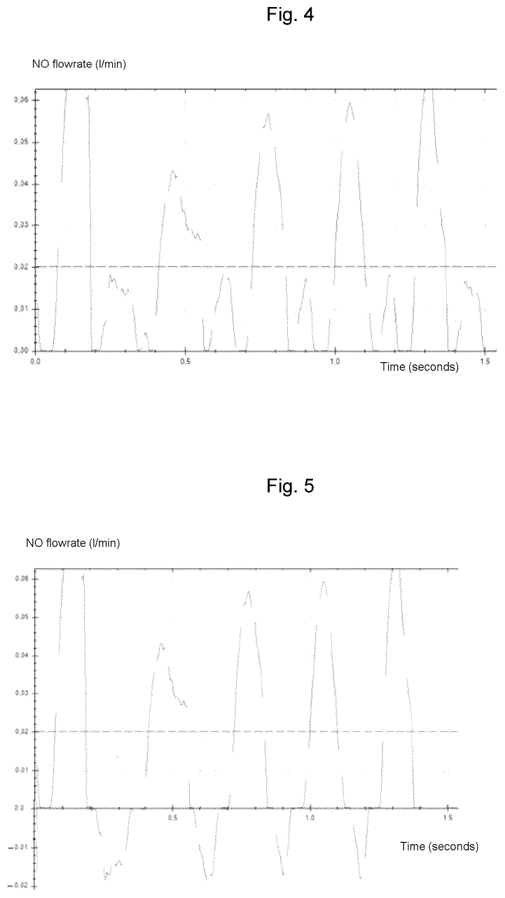

This phenomenon of saturation of the signal by “falsifying” the mean value of the NO flowrate signal operated by a “conventional” unidirectional flowrate sensor is illustrated in FIG. 3 and FIG. 4.

More precisely, FIG. 3 and FIG. 4 illustrate the influence of the vibrations that may occur during transportation, for example by ambulance or helicopter, of a patient to whom NO gas is supplied using an NO supply apparatus 1 as described above, which is equipped with a conventional unidirectional flowrate sensor 230 allowing the flow of NO to be measured only in one direction of flow of the gas, namely in the direction of the patient (i.e. arrow F in FIG. 2).

FIG. 3 shows the NO flowrate curve (l/min) regulated for a constant NO flowrate setpoint of 20 ml/min, which is a typical case of use when NO is supplied to the patient bed, i.e. to a hospitalized and bedridden patient, hence in the absence of vibrations.

As can be seen, the flowrate curve is stable over time (in seconds) and fluctuates very little around the 20 ml/min value, because the NO supply apparatus is not in motion and is therefore not subjected to vibrations or the like. In such a case of use, the pressure measurements performed by the conventional, i.e. unidirectional, pressure sensor 230 are not disturbed, on account of the absence of any vibration.

Furthermore, FIG. 4 shows, by way of comparison, the NO flowrate curve obtained under the same conditions (i.e. setpoint flowrate of 20 ml/min) but when the NO supply apparatus 1 is subjected to vibrations, such as the ones it may be subjected to during movement of the patient in the hospital, for example when going to perform a scan or the like, or even more notably during transport of the patient in a land or air vehicle, for example during transfer from one hospital to another.

As can be seen, in this case, the amplitude of the variations in NO flowrate (i.e. the flowrate measurements) measured by the conventional unidirectional sensor 230 can greatly exceed the amplitude of the NO setpoint, i.e. the fluctuations around the NO flowrate value fixed at 20 ml/min, shown in FIG. 3.

In this case, saturation of the signal is observed at 0 l/min, which “falsifies” the mean value of the NO flowrate signal. This misleads the regulation by the operating means 210, which will cause closure of the proportional solenoid valve 220 used to control the flowrate of NO and will stop the delivery of NO to the patient, with the risk of producing an undesirable rebound effect in said patient.

In order to solve this problem, according to the invention, it is proposed to use a special flowrate sensor 230 within the circuit 200 in order to be able to measure NO flowrates in both directions of flow of the gas, that is to say in the direction of the arrow F and in the opposite direction, and this despite the fact that the flow of gas is theoretically only in one direction within the gas circuit 200, namely in the direction towards the patient, that is to say the direction of the arrow F in FIG. 2. More precisely, in order to maintain what is called a “linear” mode of regulation, the special flowrate sensor 230 usable in a NO delivery apparatus 1 according to the invention is a bidirectional flowrate sensor.

In other words, according to the invention, a bidirectional flowrate sensor 230 has been inserted into the gas circuit 200 and tested under the same conditions as for FIG. 4. FIG. 5 shows the NO flowrate curve obtained for a setpoint flowrate of 20 ml/min and an apparatus 1 subjected to vibrations, with the NO supply apparatus 1 carrying such a bidirectional flowrate sensor 230.

It will be seen that the bidirectional flowrate sensor 210 has measured negative flows corresponding to the curve portions situated below the X-axis, i.e. for flowrate values of less than 0.0 ml/min.

These negative flowrates were erroneously considered as positive or zero flowrates when measured by the conventional sensor used in the test illustrated in FIG. 4 and led to erroneous operation of the solenoid valve 220 by the operating means 210 which received and processed these erroneous flowrate measurements.

However, by virtue of the bidirectional flowrate sensor 230 used instead of the conventional unidirectional sensor, this phenomenon is avoided by taking into account the negative flows generated by the vibrations caused during transport of the apparatus 1 and of the patient P.

In fact, shocks or vibrations cause symmetrical variations in the flowrate signal, i.e. positive and negative. Consequently, by taking into account the negative flowrates, the mean value of the signal is then correct because the positive and negative alternations generated by the vibrations cancel each other out.

This therefore enables the operating means 210 to better control the proportional solenoid valve 220 by avoiding closing it when this is not necessary, thanks to a coherent mean value.

A gas administration installation 100 including an NO delivery apparatus 1 according to the invention can be used to administer nitric oxide (NO), i.e. the final NO/O₂/N₂ mixture obtained, by inhalation to persons, i.e. patients, suffering from acute pulmonary arterial hypertension, in particular to dilate their pulmonary vessels and increase their oxygenation by improving pulmonary gas exchange, in particular to treat persistent pulmonary arterial hypertension of the newborn (PPHN), acute respiratory distress syndrome (ARDS) observed mainly in adults, or pulmonary hypertension (PH) in cardiac surgery in adults or children.

What is claimed is:

1. Apparatus (1) for delivering NO-containing gas, comprising:
an internal gas circuit (200) for conveying a flow of NO-containing gas, comprising flowrate control means (220, 230, 240) configured to control the flow of NO-containing gas within the gas circuit (200), said flowrate control means (220, 230, 240) comprising a proportional solenoid valve (220) and a flowrate measurement device (230), and
operating means (210) cooperating with the flowrate control means (220, 230, 240) to authorize, prohibit or adjust the flowrate of gas within said gas circuit (200),
characterized in that the flowrate measurement device (230) comprises a bidirectional flowrate sensor configured to:
perform positive and negative flowrate measurements of the NO-containing gas flow circulating in the gas circuit (200), and
supply said flowrate measurements to the operating means (210), said operating means (210) processing the positive and negative flowrate measurements from the flowrate measurement device (230) in order to operate the proportional solenoid valve (220).

2. Apparatus according to claim 1, characterized in that the operating means (210) are configured to operate the proportional solenoid valve (220) on the basis of an average flowrate value taking into account the negative flowrates supplied by the bidirectional flowrate sensor.

3. Apparatus according to claim 1, characterized in that the flowrate measurement device (230) is arranged in the gas circuit (200) upstream of the proportional solenoid valve (220).

4. Apparatus according to claim 1, characterized in that the operating means (210) comprise at least one microprocessor (211).

5. Apparatus according to claim 1, characterized in that the proportional solenoid valve (220) and the flowrate measurement device (230) form all or part or a mass flow controller (240).

6. Apparatus according to claim 1, characterized in that it comprises NO dose-setting means configured to allow a user to set a desired dose of NO.

7. Apparatus according to claim 6, characterized in that the NO dose-setting means are configured to allow a user to set an NO dose of between 1 and 80 ppmv.

8. Apparatus according to claim 1, characterized in that it further comprises a graphical display (4), and the operating means (210) are configured to order a display, on the graphical display (4), of the NO dose that is set.

9. Apparatus according claim 6, characterized in that the operating means (210) are configured to order a display, on the graphical display (4), of high and low NO alarm thresholds determined by the operating means (210) as a function of the NO dose that is set.

10. Apparatus according to claim 1, characterized in that the operating means (210) are configured to adjust or control the flowrate of the NO-containing gas flow within the circuit (200) by operating the proportional solenoid valve (210).

11. Installation (100) for supplying NO-containing gas, comprising the NO delivery apparatus (1) according to claim 1, fed with an NO/N₂ gas mixture by at least one NO/N₂ mixture source (10), and a medical ventilator (50) configured to supply a flow of respiratory gas containing O₂.

12. Installation according to claim 11, characterized in that said at least one NO/N₂ mixture source (10) comprises a gas cylinder containing an NO/N₂ gas mixture containing between 100 and 2000 ppmv of NO, the remainder being nitrogen (N₂).

13. Installation according to claim 11, characterized in that the NO delivery apparatus (1) and the medical ventilator (50) are fluidically connected to a respiratory circuit (20) in order to supply the latter with the NO-containing gas flow and the oxygen-containing gas flow and thereby obtain a combined gas mixture to be supplied to the patient.

14. Installation according to claim 13, characterized in that a flowrate sensor (25) is arranged in the respiratory circuit (20) between the medical ventilator (50) and an injection device (24) arranged in the respiratory circuit (20).

15. Installation according to either of claim 6, characterized in that the graphical display (4) comprises a touch screen and the NO dose-setting means comprise one or more tactile virtual keys actuatable by the user and displayed on the graphical display (4).
